(19) United States
(12) Patent Application Publication
(10) Pub. No.: US 2024/0316163 A1
(43) Pub. Date: Sep. 26, 2024

(54) Application of Lactoferrin in Prevention and Treatment of Alzheimer's Disease

(71) Applicant: SICHUAN UNIVERSITY, Chengdu, Sichuan (CN)

(72) Inventors: Guo CHENG, Chengdu, Sichuan (CN); Lin ZHANG, Chengdu, Sichuan (CN); Lingli ZHANG, Chengdu, Sichuan (CN)

(21) Appl. No.: 18/246,699

(22) PCT Filed: Jun. 27, 2022
(86) PCT No.: PCT/CN2022/101542

§ 371 Date: Mar. 27, 2023

(30) Foreign Application Priority Data
Sep. 26, 2021 (CN) 202111131212.X

(51) Int. Cl. A61K 38/40

(52) U.S. Cl. CPC A61K 38/40

(57) ABSTRACT

Lactoferrin (Lf) is used in the prophylaxis and treatment of Alzheimer's disease (AD), and is used as an active agent in the preparation of drugs for preventing or treating AD. By designing an in vitro model of AD, it has been proved that Lf can reduce the cell damage caused by Aβ, and play the role of anti-Alzheimer's disease by improving the anti-inflammatory ability, inhibiting the inflammation and regulating apoptosis. Meanwhile, using the APP/PS1 transgenic mouse model, it has been confirmed that Lf can reduce the phosphorylation level of Tau protein, shorten the time for mice to escape from the water maze, and improve learning and cognitive functions.

### TECHNICAL FIELD

The present invention relates to the field of drugs for the prophylaxis and treatment of Alzheimer's disease, and in particular to the use of lactoferrin in the preparation of drugs for preventing or treating Alzheimer's disease.

### BACKGROUND TECHNOLOGY

Alzheimer's disease (AD) is one of the most common neurodegenerative diseases, and its main pathological features are senile plaques (SPs) formed by excessive deposition of ß amyloid protein (Aβ) outside neurons and neurofibrillary tangles (NFTs) formed by hyperphosphorylation of Tau protein in neurons, that lead to a series of symptoms such as progressive memory loss and cognitive impairment. However, due to the complex etiology of AD, the pathogenesis of the disease is still unclear. The “amyloid cascade hypothesis” and “inflammation hypothesis” are both widely recognized mechanisms in the current scientific community. Both hypotheses suggest that the abnormal deposition of Aβ in the brain of AD patients directly or indirectly acts on neurons and neuroglia cells by a series of cascade reactions such as free radical reactions, mitochondrial oxidative damage and inflammatory reactions, and eventually leads to dysfunction or death of neurons, causing cognitive impairment and memory decline.

AD not only seriously affects the health and quality of life of patients, but also the happiness of the whole family. AD is clinically characterized by comprehensive dementia including memory impairment, anepia, apraxia, agnea, impairment of vision-spatial skill, executive dysfunction, as well as changes of personality and behavior. In the early stages of AD, patients with milder forms of dementia are mainly characterized by recent memory deficits, that is, they may forget the latest events; by temporal disorientation, that is, they are unable to distinguish time, place, direction, and so on; by still having the ability to do some familiar daily work, but having difficulties in understanding new things and dealing with complex problems; by apathy, irritability, lack of speech, and occasional inability to call the name of some things. Moderate patients are mainly characterized by severe impairment of long and short term memory, that is, they can't remember what happened a long time ago; by temporal and place disorientation, dyscalculia, anepia, apraxia, agnea; by inability to do outdoor activities independently; by needing help in dressing and personal hygiene; by often impatient and restless, constantly moving back and forth, and urinary incontinence. Severe patients will have serious loss of memory, and only some fragments of memory can be remained; the patients can't take care of themselves in their daily life, have incontinence, and need to rely on others' care completely. In addition, their limbs are stiff, and the patients are silent and will finally slip into coma.

It is of great social significance to research and develop drugs that can prevent or treat AD. In view of this, the present invention is proposed.

### Content of the Invention

The objective of the present invention is to provide an active substance for the prophylaxis and treatment of AD, which can be used in the preparation of drugs for preventing and treating AD.

For the above objectives of the present invention, lactoferrin (Lf), as an active substance, is used in the preparation of drugs for the prophylaxis and treatment of AD. The specific technical contents are:

Lf is used in the preparation of drugs for the prophylaxis of AD.

Lf is used in the preparation of drugs for the treatment of AD.

In the drugs for the prophylaxis and treatment of AD, the concentration of Lf is generally 2.5-120 μM Lf/g, and preferably 20-80 μM Lf/g.

Lf is an iron-binding glycoprotein composed of amino acids, which mainly exists in mammalian milk. It has been shown that Lf has many biological functions, including immune regulation, anti-inflammatory, and iron chelation. It has also been found that only a few neurons in the brain of young people express Lf, but a large number of neurons and glial cells in the brain of old people express Lf, suggesting that Lf may play an important role in brain aging. However, whether Lf can protect neurons from damage of AD by inhibiting inflammatory reactions has been systematically studied by the present inventors.

By designing an in vitro model of AD, the present invention has proved for the first time that Lf can reduce the cell damage caused by Aβ, and play the role of anti-Alzheimer's disease by improving the anti-inflammatory ability, inhibiting the inflammation and regulating apoptosis. At the same time, using the APP/PS1 transgenic mouse model, the present invention has confirmed that Lf can reduce the phosphorylation level of Tau protein, shorten the time for mice to escape from the water maze, and improve learning and cognitive functions. Therefore, in the present invention, it has been proved that Lf can be used for the prophylaxis and/or treatment of neurodegenerative diseases, thereby providing evidences for developing and expanding the pharmaceutical effect of Lf.

At present, the pathogenesis of AD is not clear. The present invention focuses on the effect of inflammatory reaction on AD. In the present invention, using N2a cells as the model, the effect of Lf on the protein expression and the phosphorylation level of cytokines (TNF-α, IL-6, IL-1β, etc), p38, JNK, and ERK has been analyzed; the effect of Lf on AD-related protein Tau and its phosphorylation level has been analyzed; meanwhile, different concentrations of Lf were used to act on the cell model, to study the dependence relationship between its neuroprotective effects and its concentrations, that has been used to screen effective concentrations for the prophylaxis and/or treatment of AD. The APP/PS1 transgenic mice, as the model of AD, have been used to analyze the effects of Lf on the inflammatory reaction, learning ability and cognitive function of mice brain, providing new research ideas for the prophylaxis and/or treatment of AD.

### DESCRIPTION OF FIGURES

In order to more clearly elucidate the examples of the present invention or the solutions in the prior art, the figures needed in the description of the examples or the prior art will be briefly illustrated in the following.

FIG. 1. Detecting the toxic effect of Lf on normal N2a cells by CCK8 assay;

FIG. 2. Measuring the effect of Lf on the N2a cell viability induced by Aβ₂₅₋₃₅ using CCK8 assay;

FIG. 3. The effect of Lf on N2a cell apoptosis induced by Aβ₂₅₋₃₅;

FIG. 4. The effect of Lf on inflammatory factors induced by Aβ₂₅₋₃₅ in N2a cells;

FIG. 5. The effect of Lf on the activation of TLR4/NFκB/IκBα signal pathway;

FIG. 6. The effect of Lf on AD-associated proteins in N2a cells induced by Aβ₂₅₋₃₅;

FIG. 7. The effect of Lf on the cognitive function and learning ability of APP/PS1 transgenic mice;

FIG. 8. The effect of Lf on Aβ protein, Tau protein and their phosphorylation in APP/PS1 transgenic mice.

### EXAMPLES

By following specific examples, the embodiments of the present invention were further illustrated. However, the following examples were only intended to better describe and explain the present invention, and should not be regarded as limiting the scope or practicing principles of the present invention. The examples of the present invention are not limited to the following.

In the present invention, lactoferrin, as the effective substance, was used to perform the application experiment on the prophylaxis and/or treatment of AD, and the experiment had confirmed that Lf had the pharmaceutical potentialities against neurodegenerative diseases.

### Example 1—Detecting the Toxic Effect of Lf on Normal N2a Cells by CCK8 Assay

Experimental materials: mouse neuroblastoma cells N2a, purchased from the National Collection of Authenticated Cell Cultures, the Chinese Academy of Sciences, in Shanghai.

Experimental procedures: N2a cells in logarithmic phase were inoculated into a 96-well plate at a cell density of 5×10³/well, and 6 repeated wells were set for each group. The corresponding pre-treatment was carried out according to the requirements of different experimental groups after the cells adhered to the wall. The blank group received DMEM complete culture medium without any cells; the control group received the cell culture medium without Lf; the experimental groups received the cell culture medium containing 2.5, 5, 10, 20, 40, 80 and 160 μM of Lf, respectively.

After 24 h treatment, the original culture medium was removed, and then the mixed solution of CCK8 and culture medium in a volume ratio of 1:9 was added into each well. The plate was incubated in dark for 2 h. The absorbance value of each well was measured by a microplate reader at the wavelength of 450 nm with an enzyme marker, and then the cell survival rate was calculated according to the formula. The calculation formula is:

The cell survival rate=(A_{experimental group}−A_{blank group})/(A_{control group}−A_{blank group})×100%, and A is the absorbance at the detection wavelength of 450 nm.

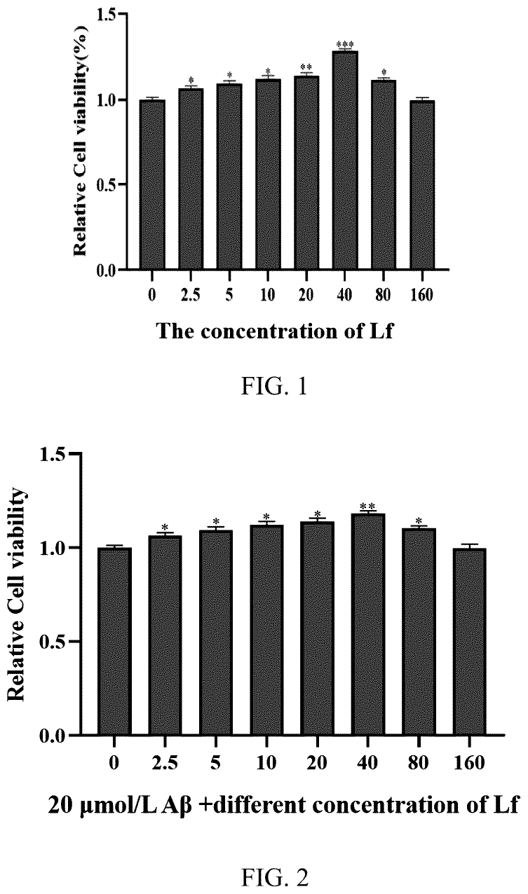

The experimental results are shown in FIG. 1. After 24 h treatment with 2.5-160 μM of Lf, the survival rate of N2a cells showed a trend of increasing and then decreasing, indicating that there was a dose-response relationship between Lf and N2a cells, in which the low concentration of Lf promoted the cell survival rate and the high concentration inhibited the cell survival rate.

### Example 2—Measuring the Effect of Lf on the N2a Cell Viability Induced by Aβ₂₅₋₃₅ Using CCK8 Assay

Experimental procedures: N2a cells in logarithmic phase were inoculated into a 96-well plate at a cell density of 5×10³/well, and 6 repeated wells were set for each group. The corresponding pre-treatment was carried out according to the requirements of different experimental groups after the cells adhered to the wall. The following treatment groups were included, that is 20 μM Aβ₂₅₋₃₅ treatment group, 20 μM Aβ₂₅₋₃₅+10 μM Lf treatment group, 20 μM Aβ₂₅₋₃₅+20 μM Lf treatment group, 20 μM Aβ₂₅₋₃₅+40 μM Lf treatment group, 20 μM Aβ₂₅₋₃₅+80 μM Lf treatment group, and 20 μM Aβ₂₅₋₃₅+160 μM Lf treatment group. The experimental groups were first treated with different concentrations of Lf for 24 h, to which were then added 20 μM Aβ₂₅₋₃₅, respectively, followed by treatment for 24 h. CCK8 assay was used to detect the cell survival rate of each group, and the detailed procedures were as described in Example 1.

The experimental results are shown in FIG. 2. Compared with Aβ₂₅₋₃₅ treatment group, the survival rate of N2a cells treated with different concentrations of Lf was significantly improved, and presented a dose-response relationship with the concentration of Lf. Thus, Lf treatment could significantly reduce the cell death caused by Aβ₂₅₋₃₅.

### Example 3—the Effect of Lf on N2a Cell Apoptosis Induced by Aβ₂₅₋₃₅

Experimental procedures: N2a cells in logarithmic phase were inoculated into a 6-well plate at a cell density of 2×10⁵/well, and the corresponding pre-treatment was carried out according to the requirements of different experimental groups after the cells adhered to the wall. The blank group Aβ₂₅₋₃₅ group, Lf group, Aβ₂₅₋₃₅+Lf group were set up. The blank group received the complete cell culture medium, without any pre-treatment. Aβ₂₅₋₃₅ group was treated with 20 μM of Aβ₂₅₋₃₅ for 24 h. In Lf groups, N2a cells were treated with 40 μg/ml of Lf for 24 h and then with 20 μM of Aβ₂₅₋₃₅ for 24 h. After treatment with the drug, the cells in each well were digested with trypsin. The plate was centrifuged at 1500 rpm for 3 min at room temperature, the supernatant was removed, and N2a cells were collected. The cells were washed twice with PBS. According to the instructions of Annexin V-FITC/PI apoptosis detection kit, an appropriate amount of binding buffer was added to suspend the cells. At room temperature, Annexin V-FITC dye was added, and then the cells were incubated for 5 min, followed by addition of PI dye. The cell solution was fully mixed, and the apoptosis rate was detected and calculated by flow cytometer. Each sample was repeated 3 times. Finally, the flow cytometry software was responsible for the data reprocessing.

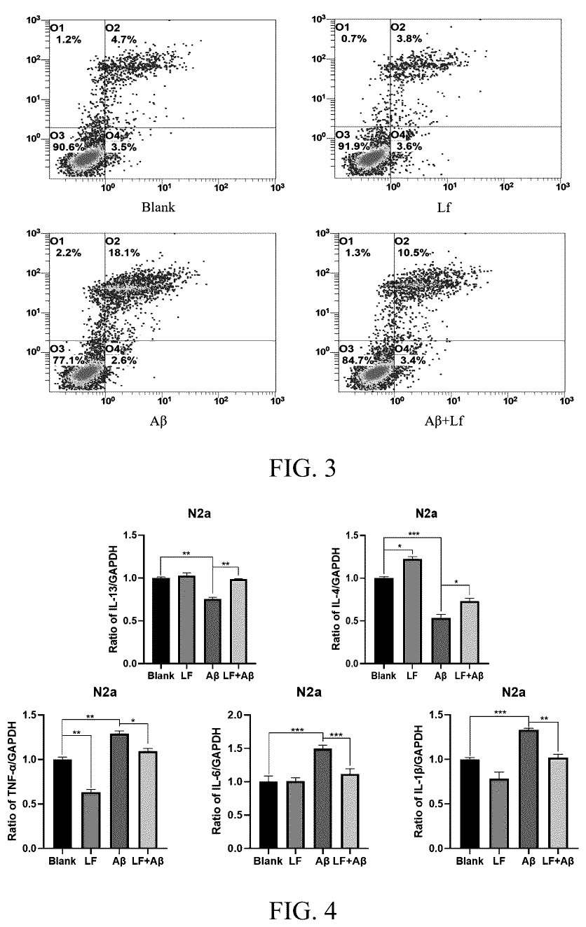

The experimental results are shown in FIG. 3. The cell apoptosis rate of blank group was 8.2%, and that of Lf group was 7.4%, which has no significant difference from that of intervention control group; compared with the intervention control group, the apoptosis rate of Aβ₂₅₋₃₅ group increased significantly, which was 20.7%; after pre-treatment with Lf, the apoptosis rate of Lf+Aβ₂₅₋₃₅ group decreased to 13.9%. The results showed that Lf could reduce the cell apoptosis induced by Aβ₂₅₋₃₅, and relieve the cytotoxicity caused by Aβ₂₅₋₃₅.

### Example 4—the Effect of Lf on N2a Cell Inflammatory Factors Induced by Aβ₂₅₋₃₅

Experimental procedures: Experimental groups were set up according to Example 3. The total RNA of cells in each group was respectively extracted with RNA extraction kit after the cells in each group were pretreated, and after determining its concentration, the total RNA was reversely transcribed into cDNA. After diluting in a ratio of 1:5-10, the cDNA was subjected to PCR amplification, and using GAPDH as the internal reference, the mRNA expression levels of IL-4, IL-6, IL-1β, IL-1β, and TNF-α were detected. Each sample was repeatedly detected three times, and the relative gene expression was calculated by using 2^{-ΔΔ}^{^{Ct}} method. The primer sequence is shown in the following table.

Primer name and sequence

| Gene | Upstream | Downstream |
|---|---|---|
| IL-4 | AGTTGTCATCCTGCTCTTCTTTC | CGAGTAATCCATTTGCATGAT |
| | TC | G |
| IL-6 | TAGTCCTTCCTACCCCAATTTCC | TTGGTCCTTAGCCACTCCTTC |
| IL-13 | AGCATGGTATGGAGTGTGGACCT | CAGTTGCTTTGTGTAGCTGAG |
| | G | CAG |
| IL-1β | GCAACTGTTCCTGAACTCAACT | ATCTTTTGGGGTCCGTCAACT |
| TNF-α | CCCTCACACTCAGATCATCTTCT | GCTACGACGTGGGCTACAG |
| GAPDH | GGATTTGGTCGTATTGGG | TCGCTCCTGGAAGATGG |

The experimental results are shown in FIG. 4. Compared with the blank group, the mRNA expression of pro-inflammatory factors TNF-α, IL-6, and IL-1β in the cells of Aβ₂₅₋₃₅ group was significantly increased, while the mRNA expression of the anti-inflammatory factors IL-4 and IL-13 was significantly decreased. Compared with Aβ₂₅₋₃₅ group, Lf treatment could significantly down-regulate the mRNA expression of pro-inflammatory factors and up-regulate the mRNA expression of anti-inflammatory factors. Similarly, as shown in FIG. 4 compared with the blank group, the mRNA expression of the pro-inflammatory factors TNF-α, IL-6, IL-1β in the cells of MPP⁺ group was significantly increased, while the mRNA expression of anti-inflammatory factors IL-4 and IL-1β was significantly decreased. Compared with MPP⁺ group, Lf treatment could down-regulate the mRNA expression of pro-inflammatory factors and up-regulate the mRNA expression of anti-inflammatory factors. The results showed that Lf could reduce the neuroinflammation induced by Aβ₂₅₋₃₅.

### Example 5—the Effect of Lf on the Activation of TLR4/NFκB/IκBα Signal Pathway

Experimental procedures: TLR4/NFκB/IκBα pathway could be activated under a variety of harmful stimulating factors, and then induce cascade reactions mediated by various inflammatory factors. The experimental groups and drug treatment were set according to Example 3. After cell treatment, the nuclear and cytoplasmic protein extraction kit was used to extract the nuclear protein, and then the protein was quantified by Bradford method. TLR4 protein, nuclear protein NF-κB, and cytosol protein IκBα, as well as their phosphorylation levels were detected by western blot.

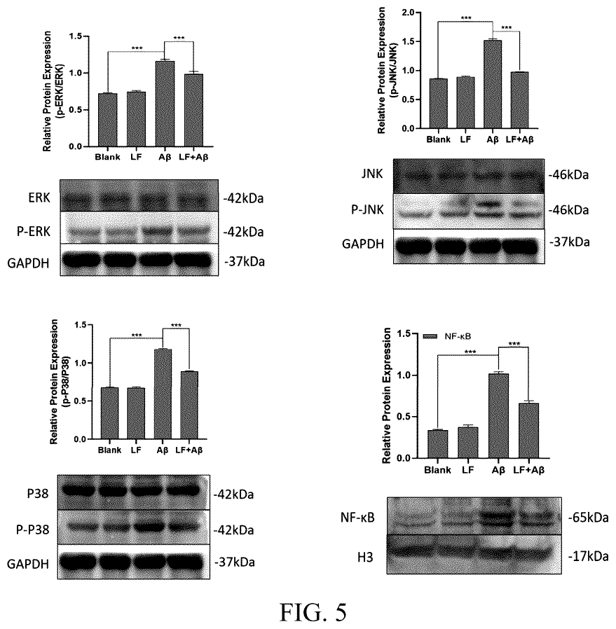

The experimental results are shown in FIG. 5. By comparing Aβ₂₅₋₃₅ group with the blank group, Aβ₂₅₋₃₅ could induce the decreased level of cytosol protein NFκB, the increased expression level of TLR4 protein and nuclear protein NFκB, and the increase in the ratio of p-IκBα/IκBα. After pre-treatment with Lf, the expression level of cytosol protein NFκB was increased, while the expression level of TLR4 and nuclear protein NFκB was decreased, and the ratio of p-IκBα/IκBα was also decreased. The results indicated that Lf could reduce the high expression of TLR4 protein and the high phosphorylation level of IκBα induced by Aβ₂₅₋₃₅ in N2a cells, inhibit the nuclear transfer of NFκB, and thus effectively inhibit the activation of TLR4/NFκB/IκBα signal pathway, thereby improving the anti-inflammation ability of cells and reducing the level of neuroinflammation.

### Example 6—the Effect of Lf on AD-Associated Proteins in N2a Cells Induced by Aβ₂₅₋₃₅

Experimental procedures: according to Example 3, experimental groups were set up, and the drug treatment, the protein extraction and the detection of the expression level of Tau protein and p-Tau were carried out.

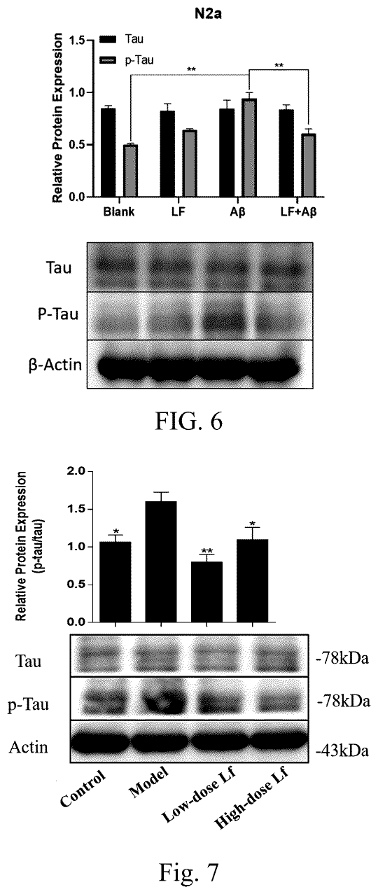

The experimental results are shown in FIG. 6. There is no significant difference in the expression level of Tau protein in each group; compared with the model group, the expression of p-Tau protein in Aβ₂₅₋₃₅ group was significantly increased; compared with Aβ₂₅₋₃₅ group, after Lf pre-treatment, the expression level of protein p-Tau decreased significantly, and the ratio of p-Tau/Tau also decreased. The results suggested that Lf could inhibit the hyper-phosphorylation of Tau protein caused by Aβ₂₅₋₃₅ in N2a cells, and reduce neurofibrillary tangles.

### Example 7—the Effect of Lf on the Cognitive Function and Learning Ability of APP/PS1 Transgenic Mice

Experimental procedures: After one week of adaptive feeding, APP/PSI transgenic mice were randomly divided into three groups: the model group (receiving normal saline), low dose Lf group and high dose Lf group, with 8 mice in each group. Eight wild-type mice were used as the control group. Low-dose Lf group and high-dose Lf group were given exogenous lactoferrin at 2 mg/kg/d and 6 mg/kg/d, respectively. The control group and the model group were given the same volume of normal saline. The water maze experiment included the place navigation test and the spatial probe test.

During the place navigation test, by repeatedly training the mice, the memory of mice to the surrounding environment was enhanced, and the mice could find the underwater escape platform in a short time. This test was used to detect the spatial memory function of mice. During the spatial probe test, according to the prompts of spatial memory, the test mice should spend more time and energy to find the quadrant of the escape platform. The quadrants were arranged clockwise, and the order was the first, second, third and fourth quadrants. The place where the platform was located was the third quadrant, which was on the opposite side of the first quadrant. The test cycle was five days. The place navigation test was carried out on the first four days, and the spatial probe test was carried out on the fifth day. This procedure was that of classic Morris water maze test.

Place navigation test: before the test, APP/PSI transgenic mice and wide type mice were put on the platform, first allowed to adapt to the environment, and the adaptation time was set to 20 s. Then, the mice were put in the water, facing the wall of the first quadrant; the time was recorded, and the recording was stopped when the mice in the model control group climbed onto the platform and stayed for 5 s. 60 s is the longest record of this experiment. If the mice could not get on the platform within the specified time, they will be actively guided to reach the platform and stay for 10 s. Finally, the mice would be cleaned. After the experiment, the mice were put into the cage. Later, follow this process, in the order of the first, second, third and fourth quadrants, the mice were allowed to walk clockwise once a day, which was lasted for 4 days. The latency time of mice on the platform in four quadrants was recorded for each experiment, and the average value of several quadrants was estimated to evaluate the spatial learning ability of mice.

Spatial probe test: on the last day of the experiment, the platform under the water surface was removed, but the test environment, water temperature and place navigation test in the water maze remained unchanged. The test mice were placed in the water facing the wall of the first quadrant, and then the swimming routes of the mice within 60 seconds were recorded and observed. The water on the mice were cleaned, and then the mice were placed in the cage. The test in other quadrants need not be carried out again. The evaluation of the spatial memory ability of mice was completed by recording the number of times the mice crossed the platform, and the ratio of the time required for the mice to reach the third quadrant to the total time.

The experimental results are shown in FIG. 7. The time for mice in the model group to escape the water maze was 120 s, while the time for mice in the control group was only 20 s, indicating that the learning ability and cognitive function of mice in the model group were significantly reduced; the time for mice in the low-dose and high-dose Lf groups to escape the water maze was shortened to 70 s and 59 s, respectively, indicating that Lf could effectively improve the cognitive dysfunction of mice with neurodegenerative diseases and improve their learning ability.

### Example 8—the Effect of Lf on Aβ Protein, Tau Protein and their Phosphorylation in SAMP-8 Mice

Experimental procedures: according to Example 7, experimental groups were set up, and pre-treatment was carried out. After pre-treatment, SAMP-8 mice were randomly divided into three groups: the model group (receiving normal saline), low dose Lf group and high dose Lf group SAMR-1 mice were used as the control group. 3 mice in each group were dissected to collect the brain tissue, which was broken by tissue homogenizer, and then the total protein of brain tissue was extracted. Aβ protein, Tau protein and their phosphorylation in mouse brain tissues were determined according to Example 5.

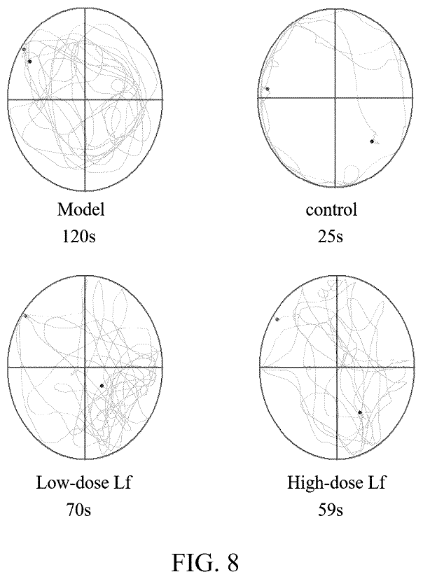

The experimental results are shown in FIG. 8. Compared with the model group, the phosphorylation level of Tau protein in the low-dose Lf group and the high-dose Lf group decreased significantly, and the ratio in both groups also decreased significantly. The results indicated that Lf could reduce the neurofibrillary tangles caused by the phosphorylation of neurofibrils in the brain of neurodegenerative mice.

As shown in the above examples 1 to 8, Lf could inhibit the phosphorylation levels of ERK, JNK and P38 proteins, reduce the expression of NF-κB protein, increase the levels of anti-inflammatory factors, lower the levels of pro-inflammatory factors, and enhance the ability of cellular anti-inflammatory response. Lf could also reduce the phosphorylation level of Tau protein in nerve cells and brain tissues of mice with neurodegenerative diseases, and improve the learning ability and cognitive function of mice. Therefore, the present invention confirmed the application potential of Lf against AD, and also provided a theoretical basis for the use of Lf in the prophylaxis and/or treatment of AD.

While the present invention had been particularly illustrated and described by reference to the specific examples, it should be recognized that many other changes and modifications could be made without departing from the spirit and scope of the present invention. Therefore, it meant that all these changes and modifications within the scope of the present invention were included in the appended claims.

1. A drug composition for prophylaxis and treatment of Alzhemier's disease, comprising lactoferrin and one or more pharmaceutically acceptable excipient.

2. (canceled)

3. The drug composition of claim 1, having a concentration of lactoferrin of 2.5-120 μM Lf/g.

4. The drug composition according to claim 3, wherein the concentration of lactoferrin is 20-80 μM Lf/g.

5. A method for prophylaxis and treatment of Alzhemier's disease, comprising administering the drug composition according to claim 1 to a subject in need thereof.
